(19) United States
(12) Patent Application Publication
(10) Pub. No.: US 2022/0238228 A1
(43) Pub. Date: Jul. 28, 2022

(54) INTELLIGENT TELEHEALTH PLATFORM

(71) Applicant: EO Care, Inc., Wellesley, MA (US)

(72) Inventors: David C. Batista, Brookline, MA (US); Stacey Batista, Brookline, MA (US); Benjamin P. Caplan, Needham, MA (US); Sean J. Collins, Wellesley, MA (US); Birch Norton, Ipswich, MA (US); Kristen P. Parton, Medfield, MA (US)

(21) Appl. No.: 17/579,436

(22) Filed: Jan. 19, 2022

Related U.S. Application Data
(60) Provisional application No. 63/140,689, filed on Jan. 22, 2021.

(51) Int. Cl. G16H 50/20; G16H 20/10; G16H 80/00; G16H 10/60

(52) U.S. Cl. CPC G16H 50/20; G16H 10/60; G16H 80/00; G16H 20/10

(57) ABSTRACT

A system and method of using an intelligent telehealth platform to generate a recommended treatment plan to treat a malady of a user. The method includes obtaining a user profile dataset associated with a user. The method includes selecting, based on the user profile dataset, a first machine learning model from a set of machine learning models respectively trained to predict cannabis treatment responses. The method includes generating, by a processing device, a recommended treatment plan for using one or more cannabis products to treat a malady of the user based on the user profile dataset and the first machine learning model. The method includes transmitting the recommended treatment plan to a client device to cause the client device to display the recommended treatment plan.

### CROSS-REFERENCE TO RELATED APPLICATIONS

This application claims the benefit of U.S. Provisional Application Ser. No. 63/140,689 entitled “INTELLIGENT TELEHEALTH PLATFORM,” filed Jan. 22, 2021, the disclosure of which is incorporated herein by reference in its entirety.

### TECHNICAL FIELD

The present disclosure relates generally to software technology, and more particularly, to systems and methods for using an intelligent telehealth platform to generate a recommended treatment plan to treat a malady of a user.

### BACKGROUND

Machine learning is a type of artificial intelligence when computers are programmed to learn information without human intervention. In machine learning, the development of the underlying algorithms rely on computational statistics. Computers are provided data and then the computers learn from that data. The data actually teaches the computer by revealing its complex patterns and underlying algorithms. The larger the sample of data the machine is provided, the more precise the machine's output becomes. Machine learning in healthcare is becoming more widely used and is helping patients and clinicians in many different ways.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1 is a block diagram that illustrates an example computing architecture, in accordance with some embodiments of the present disclosure;

FIG. 2 is a block diagram of an example intelligent telehealth (IT) platform, in accordance with embodiments of the disclosure;

FIG. 3A is an example graphical user interface of the telehealth application 160 depicting a method for displaying a list of dispensaries stored in a storage of the IT platform 200, according to some embodiments;

FIGS. 3B-3C are example graphical user interfaces of the telehealth application 160 depicting a method for displaying a list of products stored in the IT platform 200, as well as the details of each of the products, according to some embodiments;

FIGS. 3D-3E are example graphical user interfaces of the telehealth application 160 depicting a method for displaying a list of patient profiles stored in the IT platform 200, as well as the details of each of the patient profiles, according to some embodiments;

FIG. 3F is an example graphical user interfaces of the telehealth application 160 depicting a method for displaying the model outputs from each of the models of the IT platform 200, according to some embodiments;

FIGS. 3G-3H are example graphical user interfaces of the telehealth application 160 depicting a method for displaying a draft careplan created by the models of the IT platform 200, according to some embodiments;

FIG. 31 is an example graphical user interface of the telehealth application 160 depicting a method for displaying product specific instructions, according to some embodiments;

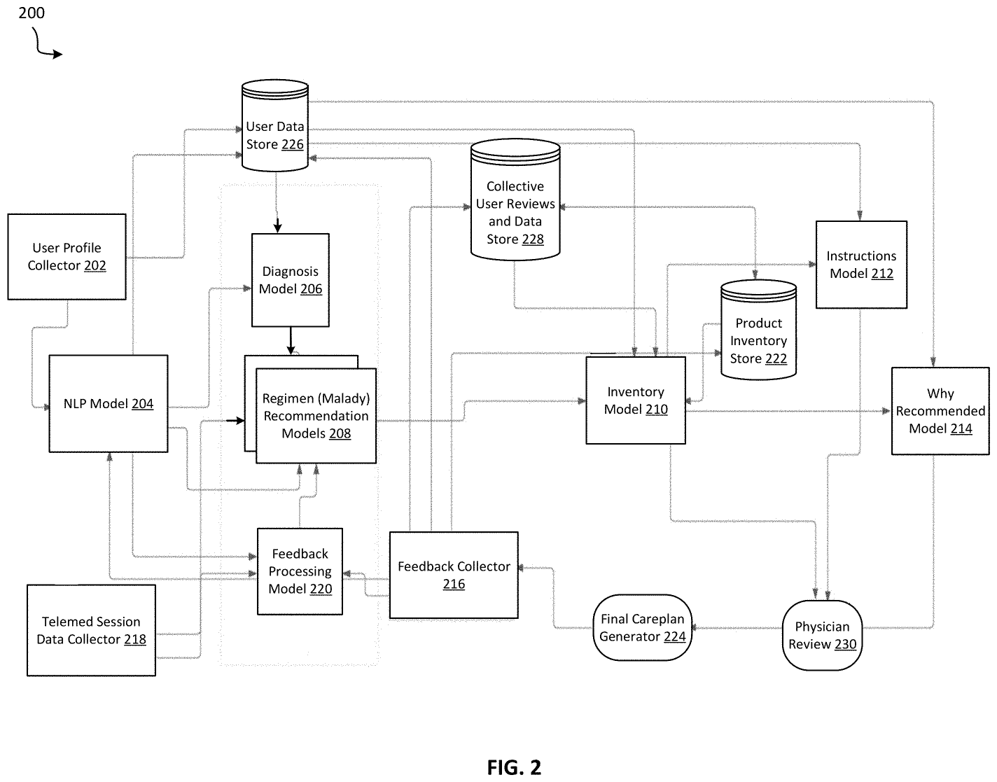

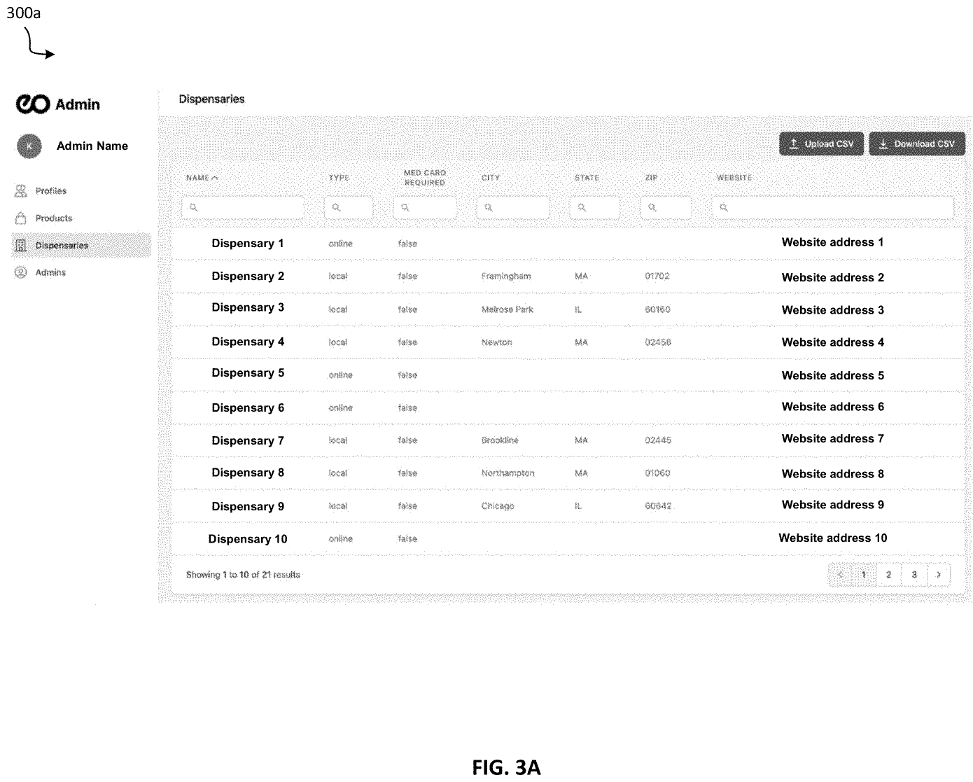

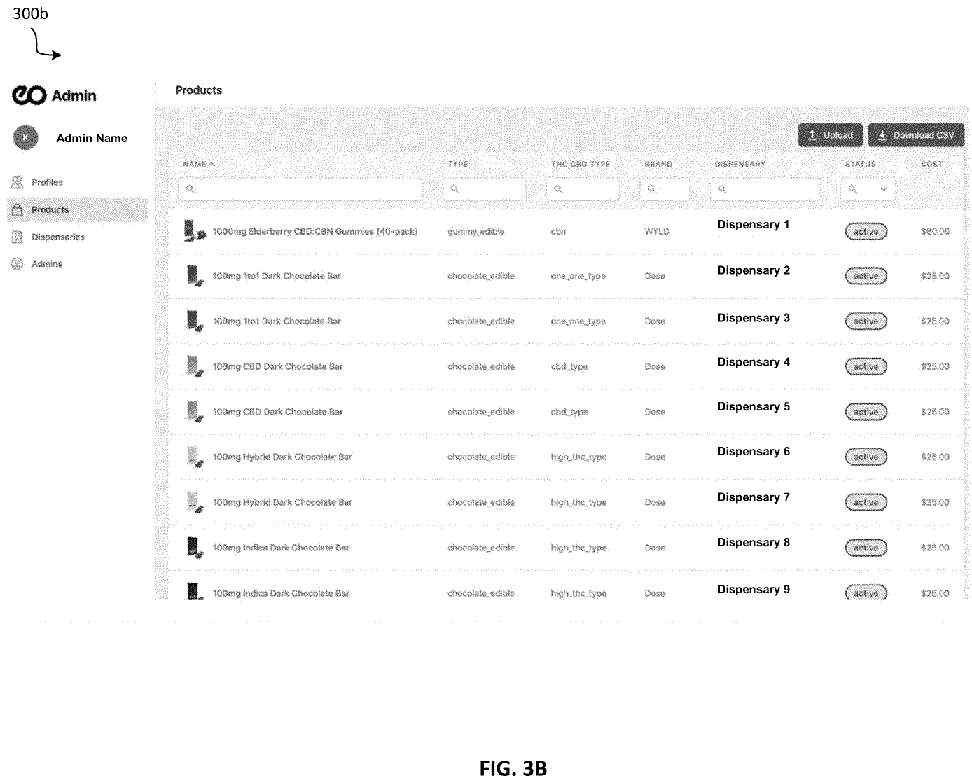

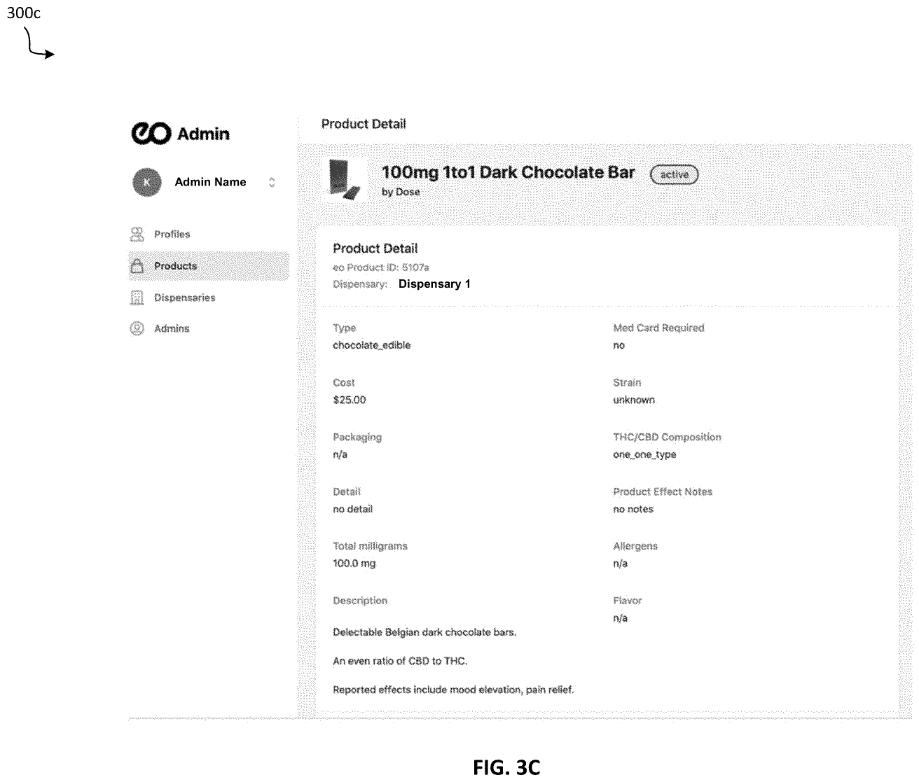

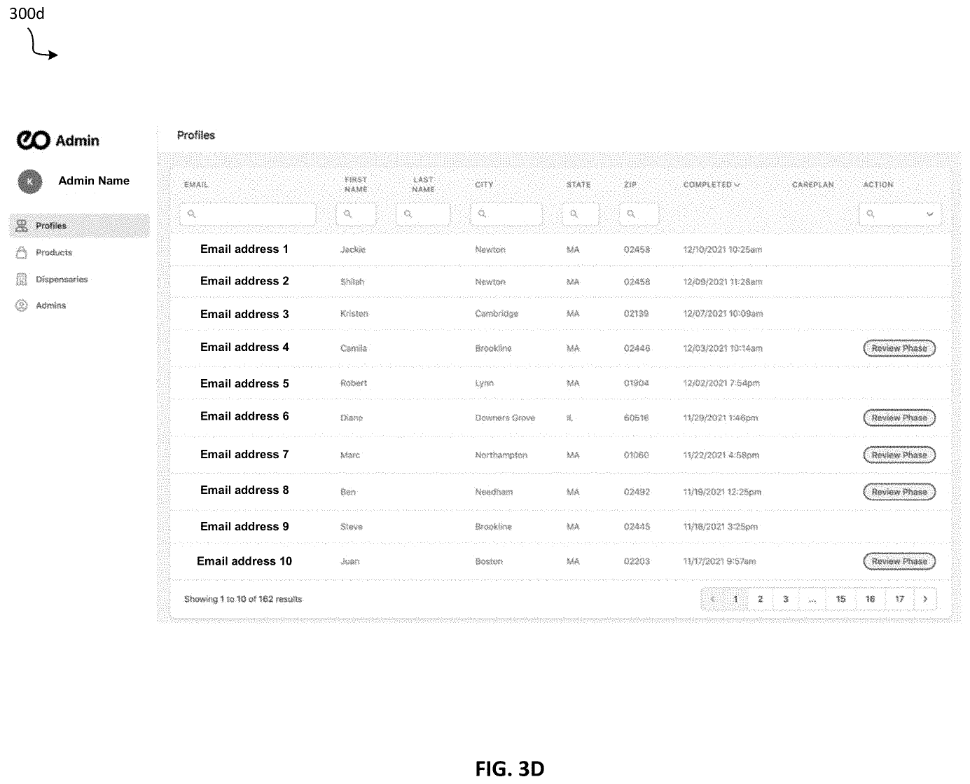

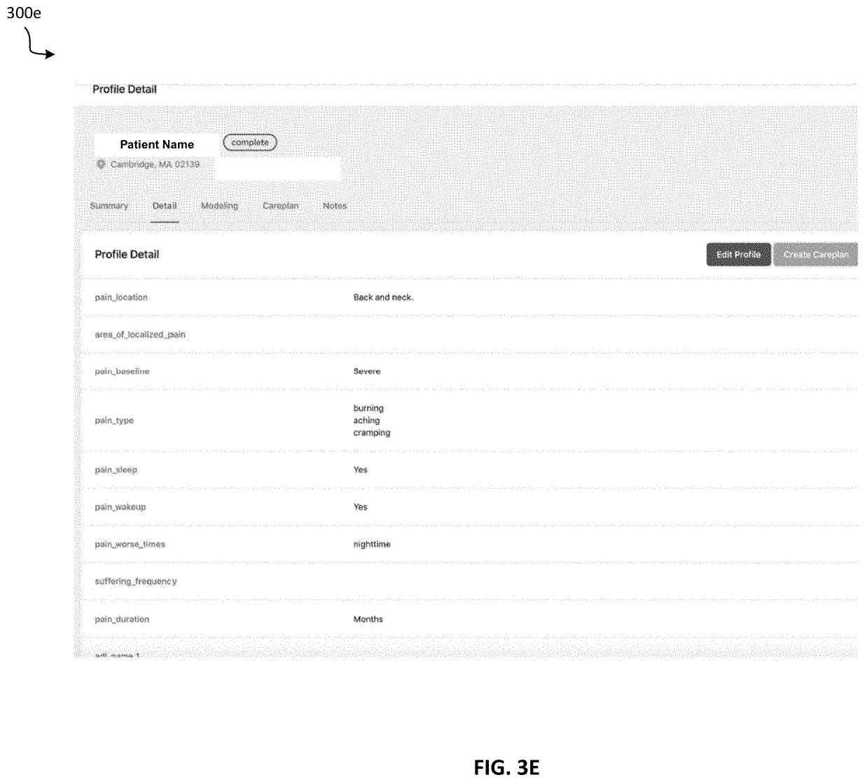

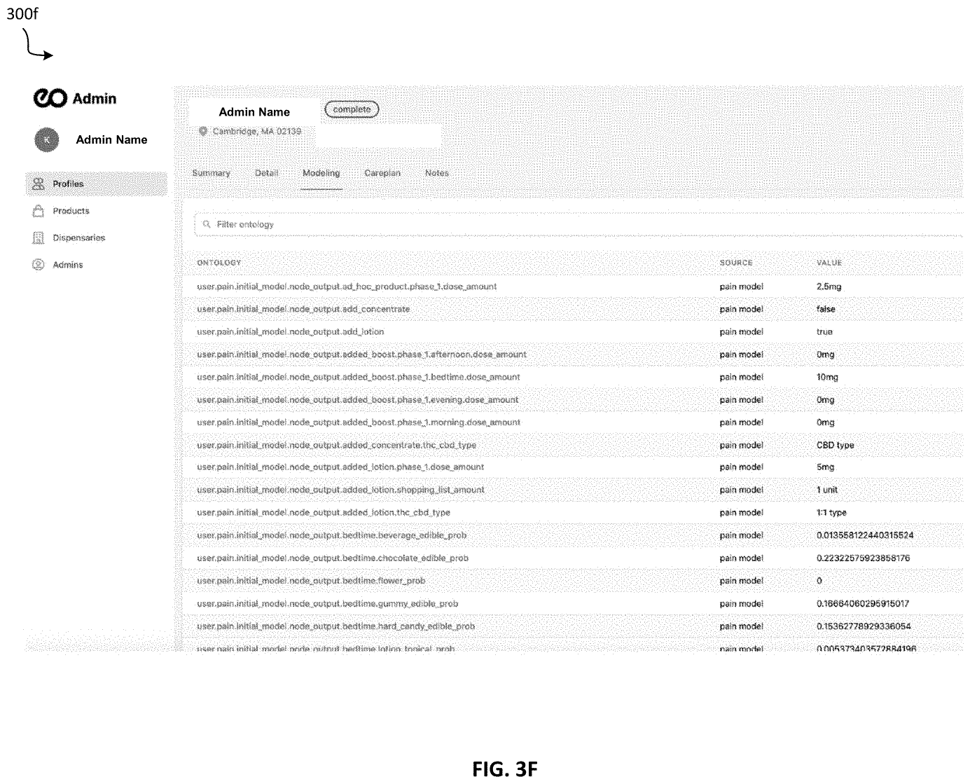

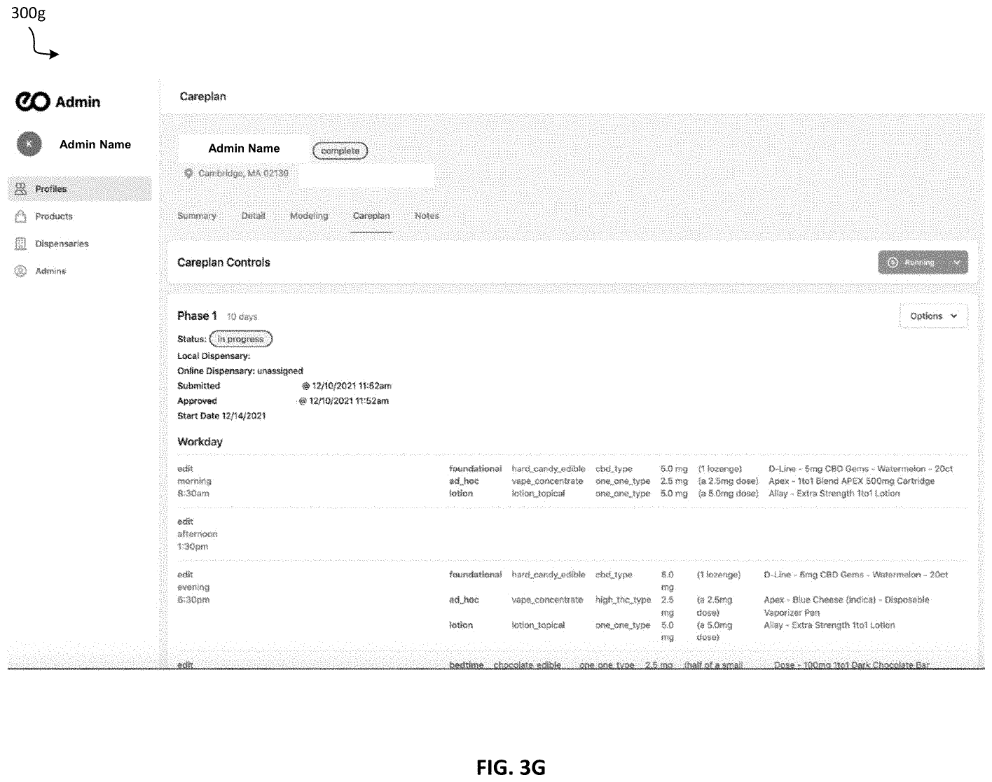

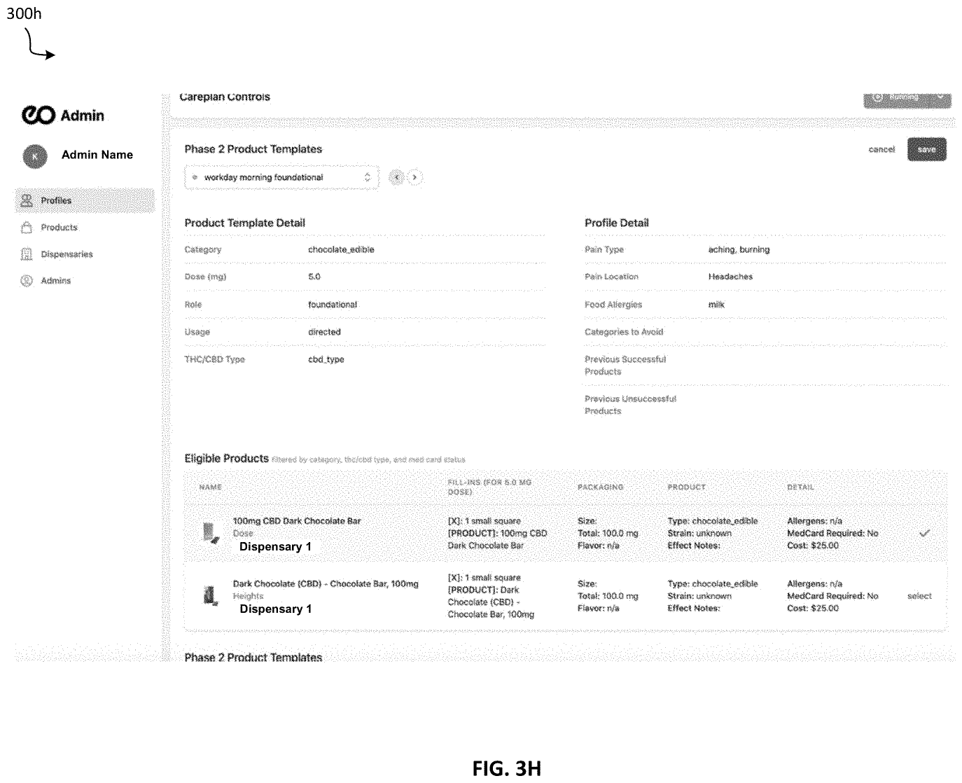

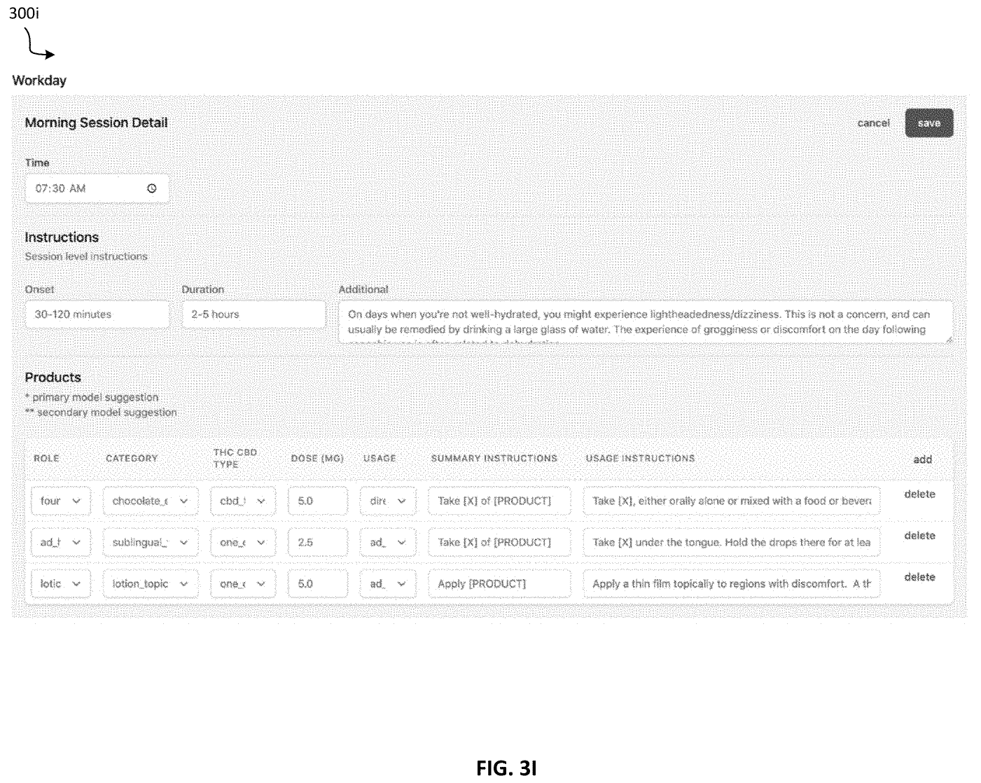

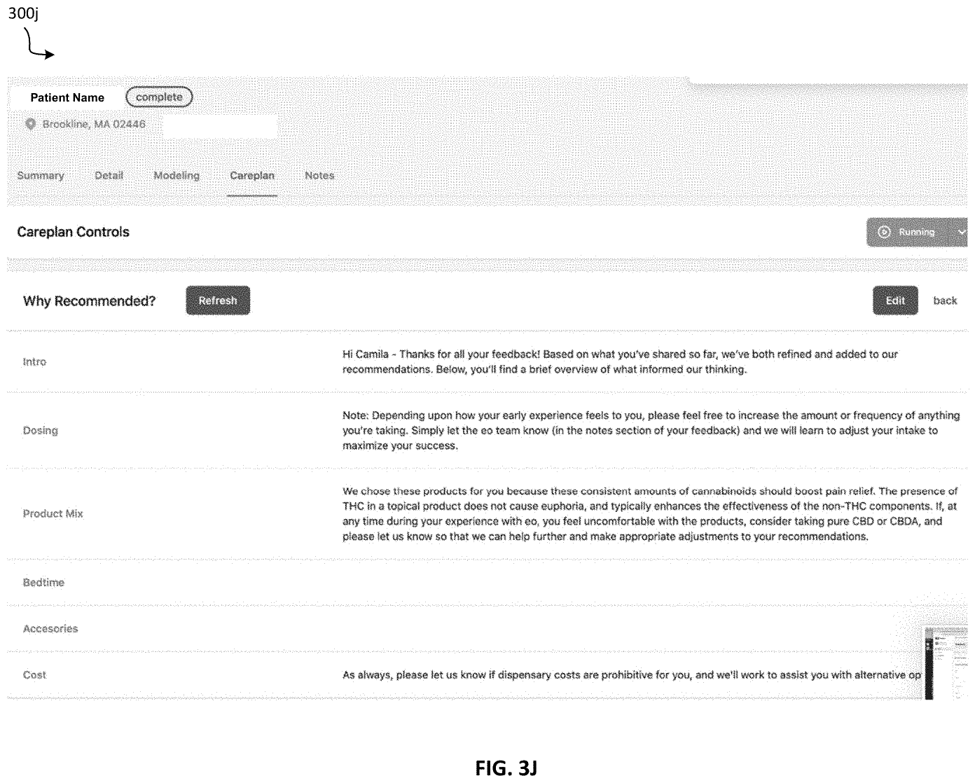

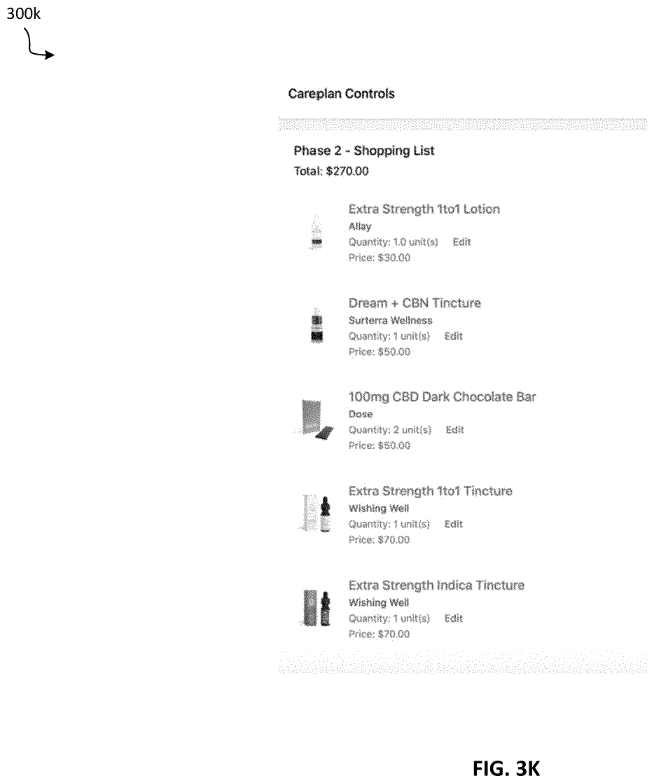

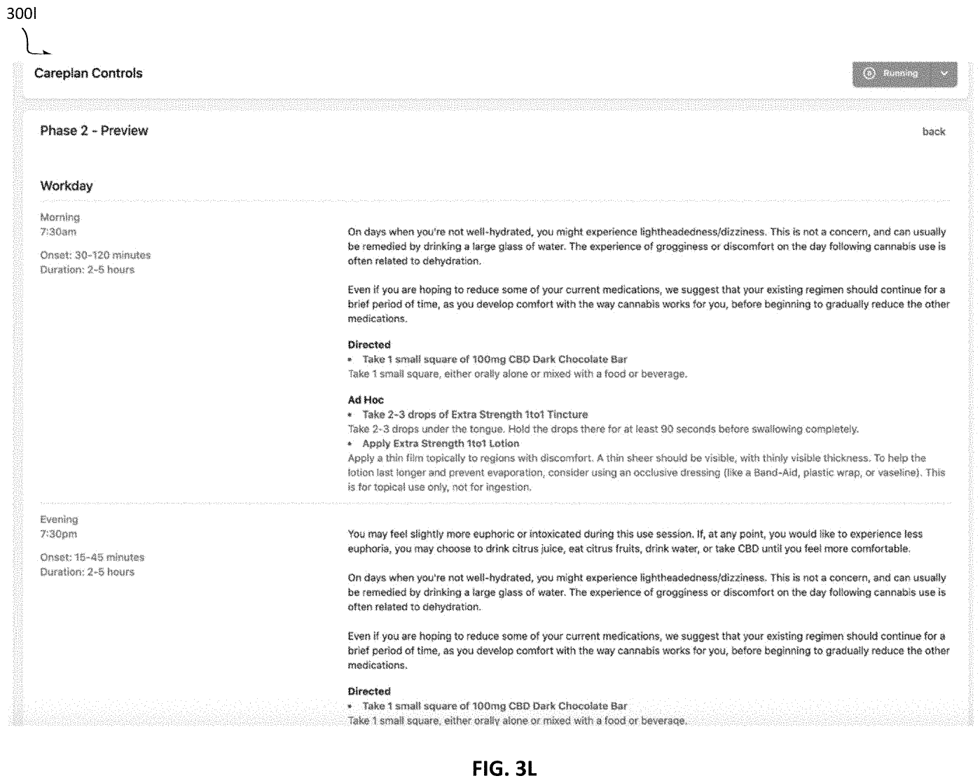

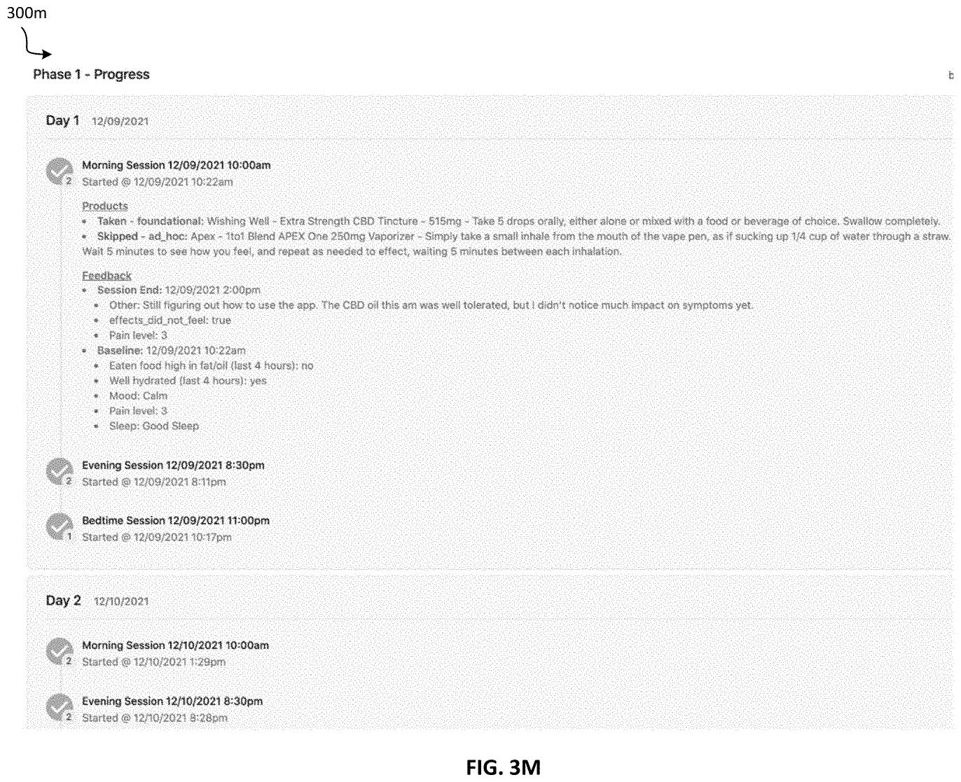

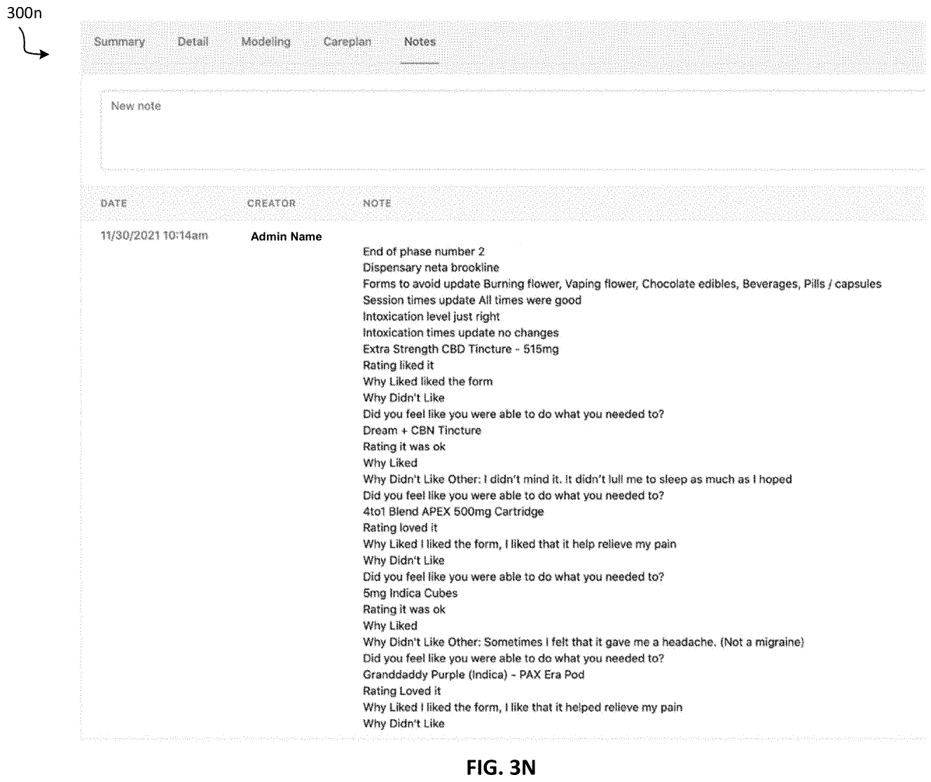

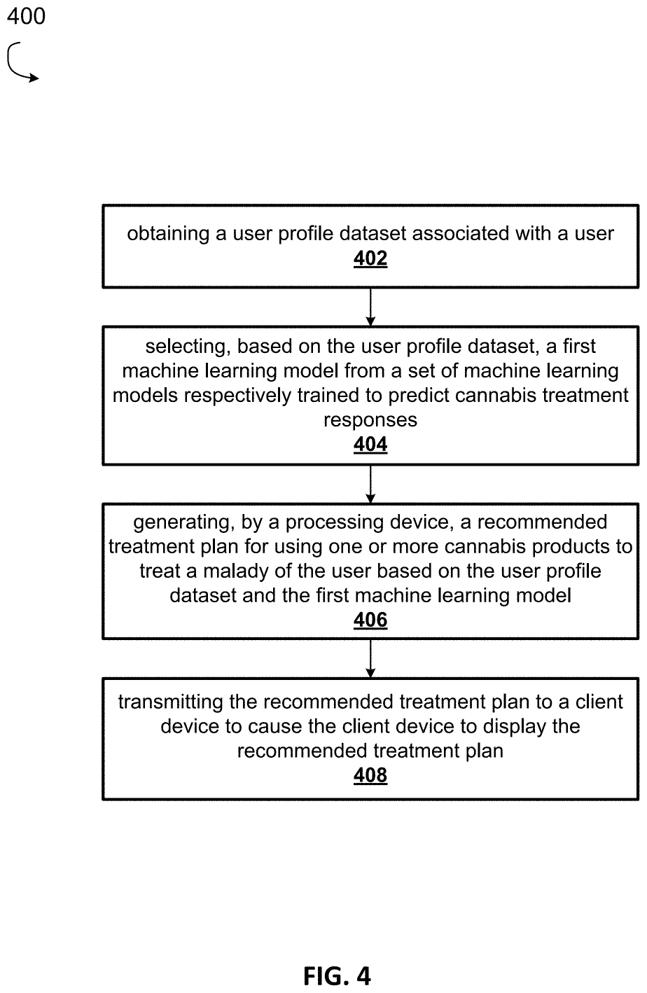

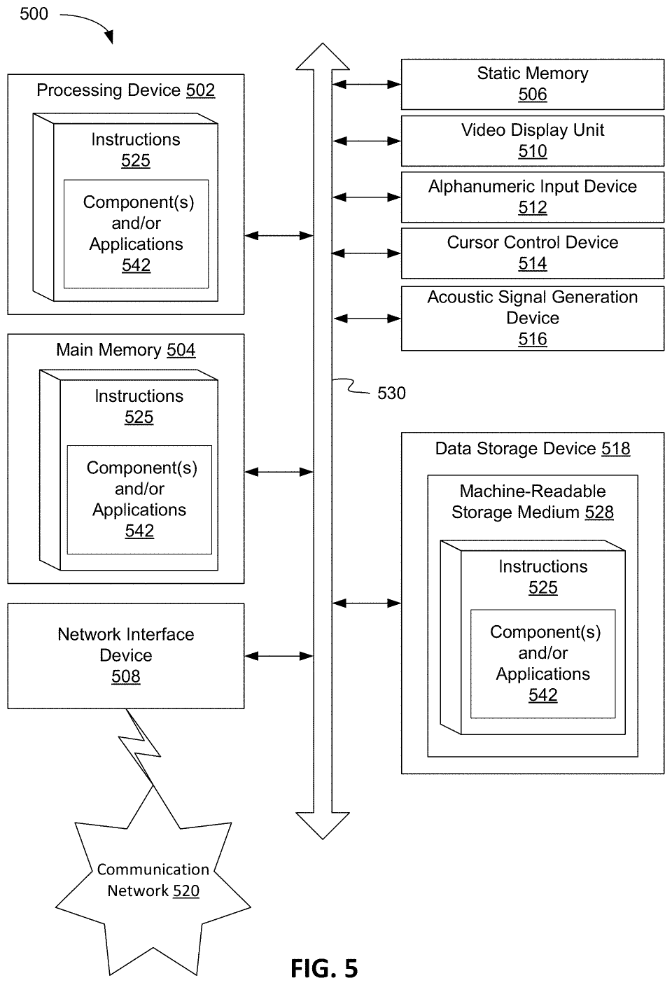

FIG. 3J is an example graphical user interface of the telehealth application 160 depicting a method for displaying the model output of the why recommendation model 214 in FIG. 2, according to some embodiments;

FIG. 3K is an example graphical user interface of the telehealth application 160 depicting a method for displaying a user's shopping list, according to some embodiments;

FIG. 3L is an example graphical user interface of the telehealth application 160 depicting a method for displaying a draft careplan created by the models of the IT platform 200, according to some embodiments;

FIG. 3M is an example graphical user interface of the telehealth application 160 depicting a method for displaying a user's progress in completing the careplan, according to some embodiments;

FIG. 3N is an example graphical user interface of the telehealth application 160 depicting a method for displaying a notes session to track any communication with the patient, survey data, or physician telemedicine (sometimes referred to as, “telemed”) notes, according to some embodiments;

FIG. 4 is a flow diagram depicting a method for using the intelligent telehealth platform 200 to generate a recommended treatment plan to treat a malady of a user, according to some embodiments; and

FIG. 5 is a block diagram of an example computing device that may perform one or more of the operations described herein, in accordance with some embodiments.

### DETAILED DESCRIPTION

An intelligent telehealth platform is described. In one embodiment, the telehealth platform described herein provides intelligent recommendations for cannabis-based treatment to an individual. Worth noting, although many of the examples described herein refer to cannabis for clarity and brevity, any other suitable treatment methods are equally contemplated herein. In one embodiment, the platform described provides a complete, end-to-end solution for individuals seeking recommendations and guidance for healthcare treatment solutions. For example, the provided embodiments describe collecting information about an individual's ailments, connecting the individual with healthcare professionals, providing customized recommendations for treatment (e.g., including pharmaceuticals), an online marketplace for custom treatment solutions, personalized real-time treatment instructions and walkthroughs, advanced custom treatment optimizations, personalized reminders, etc.

Designing an intelligent telehealth platform/system for cannabis-based treatment to an individual presents several problems. For one, the platform must be able to make accurate predictions about the user even when there is no reliable source of data to use to build a model. Furthermore, the experience of cannabis effects by individuals is often highly idiosyncratic. Thus, the problem of idiosyncratic reactions to substances like cannabis requires a novel approach to both solving for utility and speeding up the system overall.

Aspects of the present disclosure address the above-noted and other deficiencies by implementing Bayesian and Decision Networks to best provide a knowledge-engineered platform that could eventually update itself and incorporate data when it becomes available. Advantageously, the combination of traditional and Bayesian approaches used produce a unique-to-each-user projection and prediction model consuming both discrete, observed quantitative data with continuous, subjective, and intuitive inputs regarding the real-time, experienced state of the individual. The Bayesian service generates (e.g., drives) a series of “tuning” questions that begins to self-learn. It also gives a method of observing and quantifying how trustworthy and accurate a given user's feedback is, as well as a certainty measure on the model's predictions and recommendations—an important insight for health- and medicine-related decision making—that is then incorporated into the unique-to-each-user prediction model for ever-increasing accuracy and temporal range. In one embodiment of the system, all this occurs in a runtime environment enabling nearly-immediate computation with no offline processing. In comparison, current approaches to state-change management combine online information retrieval of generic effects with incomplete asynchronous, “remembered” effects from the compound in a traditional medical interview that could take weeks or even months to fully cycle, and do not incorporate individualized subjective, real-time state-change experience data.

The overall intelligence model may include multiple pieces. At a high level are the inputs of the data used by the model. Such data may include user profiling inputs, product inventory, inputs from healthcare and pharmaceutical experts, inputs the user provides based on questions the platform asks, data indicative of physician expertise and experience associated with cannabis, physician approval of the recommended treatment plan, and/or data on how the user interacts with the system.

The embodiments described herein provide for a variety of novel features, including, but not limited to: the generation of a personalized, holistic, calendar-based treatment plan spanning medicine instructions (e.g., how much of what to take, when, where and how), diet, exercise and mindfulness; the enablement of real-time, structured patient feedback using established clinical measures, data sources from other user applications, and unstructured voice and text feedback all while the patient is experiencing the effects of the medication. the enablement of end-of-day, overall well-being and satisfaction check-ins using structured, established measures and unstructured voice and text inputs; and/or the synthesis of all patient inputs and third-party data into a clinical dashboard, through which the clinician can use a series of proprietary filters and search mechanisms to efficiently track individual patient progress and determine connections and insights related to medical substances ingested, patient health profile, patient feedback and patient actions/adherence to a care program (e.g., an eo 30-day care program).

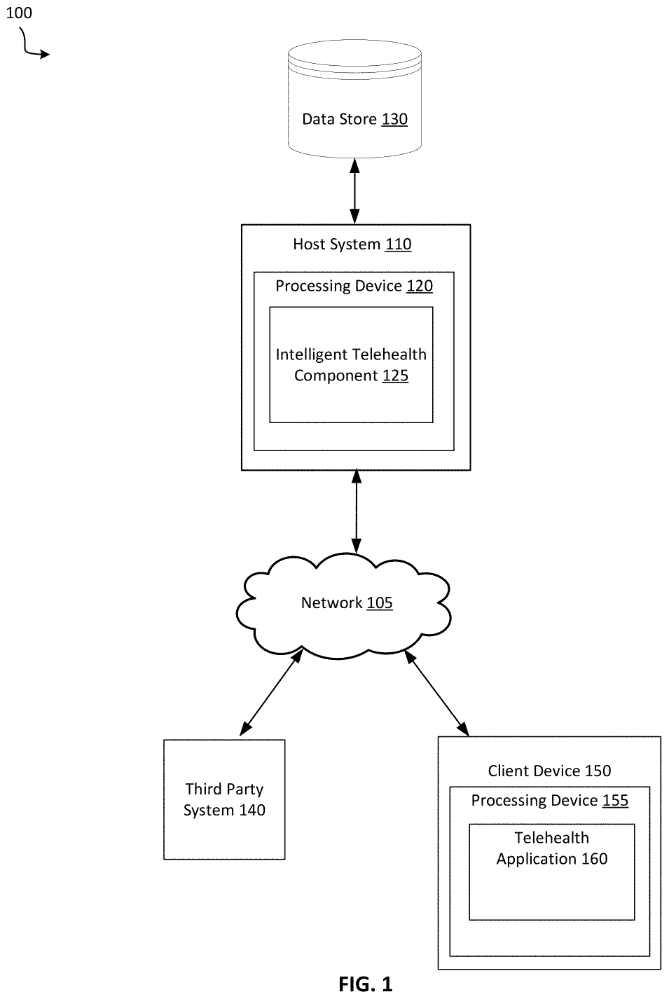

FIG. 1 is a block diagram that illustrates an example computing architecture 100, in accordance with some embodiments of the present disclosure. The computing architecture 100 may include host system 110, third-party system 140, and client device 150.

As illustrated in FIG. 1, computing architecture 100 includes host system 110 that includes computing processing device 120 and data store 130. The host system 110, third-party system 140, and client device 150 are coupled to each other (e.g., may be operatively coupled, communicatively coupled, may communicate data/messages with each other) via network 105. Network 105 may be a public network (e.g., the internet), a private network (e.g., a local area network (LAN) or wide area network (WAN)), or a combination thereof. In one embodiment, network 105 may include a wired or a wireless infrastructure, which may be provided by one or more wireless communications systems, such as a WiFi′ hotspot connected with the network 105 and/or a wireless carrier system that can be implemented using various data processing equipment, communication towers (e.g. cell towers), etc. The network 105 may carry communications (e.g., data, message, packets, frames, etc.) between the various components of host system 110.

The client device 150 includes a processing device 155 that executes a telehealth application 160 that the client device 150 displays on a computer screen (local or remote) allowing a user of the client device 150 to view and exchange data (e.g., user input data, recommendation requests, recommendations, model outputs, notifications, alerts, etc.) with any other computing devices (e.g., host system 110, third party system 140) and/or data store (e.g., data store 130) connected to the network 105. In some embodiments, an administrator may use the telehealth application (sometimes referred to as, “admin portal”) executing on a client device 150 in an administrator mode to manage and/or operate the host system 110

The telehealth application 160 may monitor interactions of a user (not shown in FIG. 1) of the client device 150 with the telehealth application 160 and/or the client device 150 to intercept and/or collect data processed by processing device 155, similar to a screen scraper, packet interceptor, application programming interface (API) hooking process, or other such application. The telehealth application 160 may intercept and/or receive data, such as mouse clicks, scroll wheel movements, gestures such as swipes, pinches, or touches, or any other such interactions. The telehealth application 160 may receive data via multiple choice, sliders, text entry, via open voice to text, etc. The telehealth application 160 may send the intercepted and/or received data to the host system 110 via network 105.

In some embodiments, the telehealth application 160 may display a GUI screen (e.g., pop-up) asking for the user's permissions to have access to data (e.g., medical data, wellness data, personality data, user interaction data, ect.) about the user from external sources, and if access is granted, retrieve the data from the external sources. For example, the IT platform 200 may ask the user for permission to access their data that was gathered by their wearable device (e.g., smart watch, headset, wireless earbuds, fitness tracker, blood pressure monitor, etc.), and if approved, retrieve the data from the wearable device (or from the user's smart phone that is tethered to the wearable device). For example, a user that listens to a variety of music via an online music service may grant the IT platform 200 permission to connect to a public application programming interface (API) of the online music service and view the user's music choice. This music-related information may be a predictor or indicator of the user's state-change. The IT platform 200 may then use the data retrieved from the external sources to perform even deeper predictions about the user, which in turn, improves the IT platform's 200 capability to generate a recommended treatment plan that is best fit for the user.

The data store 130 may be a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. In some embodiments, the data store 130 may correspond to a plurality of physically separate data stores (e.g., user data store, a collective user reviews and data store, a product inventory store, etc.) that are communicatively coupled to the network 105.

Each component may include hardware such as processing devices (e.g., processors, central processing units (CPUs), memory (e.g., random access memory (RAM), storage devices (e.g., hard-disk drive (HDD), solid-state drive (SSD), etc.), and other hardware devices (e.g., sound card, video card, etc.). The host system 110, third-party system 140, and client device 150 may include any suitable type of computing device or machine that has a programmable processor including, for example, server computers, desktop computers, laptop computers, tablet computers, smartphones, set-top boxes, etc. In some examples, host system 110, third-party system 140, and client device 150 may include a single machine or may include multiple interconnected machines (e.g., multiple servers configured in a cluster). In some embodiments, host system 110 may be part of a cloud computing system. The host system 110, third-party system 140, and client device 150 may execute or include an operating system (OS), as discussed in more detail below. The OS of a server may manage the execution of other components (e.g., software, applications, etc.) and/or may manage access to the hardware (e.g., processors, memory, storage devices etc.) of the computing device.

In some embodiments, processing device 120 may execute an intelligent telehealth (IT) component 125. In some embodiments, the IT component 125 may include at least part of a machine learning model for intelligently generating a recommended treatment plan (sometimes referred to as, “careplan” or “care plan”) to treat a malady of a user. The IT component 125 may obtain a user profile dataset associated with a user. The IT component 125 may further select, based on the user profile dataset, a first machine learning model from a set of machine learning models respectively trained to predict cannabis treatment responses. The IT component 125 may generate a recommended treatment plan for using one or more cannabis products to treat a malady of the user based on the user profile dataset and the first machine learning model. The IT component 125 may transmit the recommended treatment plan to a client device to cause the client device to display the recommended treatment plan. The IT component 125 may further transmit a request to a physician (e.g., third-party system 140) for approval of the recommended treatment plan. The IT component 125 may further receive physician approval of the recommended treatment plan prior to transmitting the recommended treatment plan to the client device 150. Further details regarding the IT component 125 are discussed at FIGS. 2-5 below.

In some embodiments, the data store 130 may include a repository of one or more pieces of user profile data, feedback data, product inventory data, collective user reviews, telemed session data, and careplans.

Although FIG. 1 shows only a select number of computing devices (e.g., client devices 150, third party system 140) and data store (e.g., data store 13), the computing architecture 100 may include any number of computing devices that are interconnected in any arrangement to facilitate the exchange of data between the computing devices.

FIG. 2 is a block diagram of an example intelligent telehealth (IT) platform 200, in accordance with embodiments of the disclosure. In some embodiments, at least a portion of the IT platform 200 may be implemented by the IT component 125 in FIG. 1. The IT platform 200 (sometimes referred to as, “platform” hereafter) may include and/or execute a user profile collector 202, a nature language processing (NLP) 204, a diagnosis model 206, one or more regimen recommendation models 208, an inventory model 210, an instructions model 212, a why recommended model 214, a feedback collector 216, a telemed session data collector 218, a feedback processing model 220, and a final careplan generator 224. In some embodiments, the diagnosis model 206, the regimen recommendation models 208, and/or the feedback processing model 220 may be combined into a single model that performs the combined functionality of each the models. In some embodiments, each of the models (e.g., diagnosis model 206, the regimen recommendation models 208, and/or the feedback processing model 220) may be split into a plurality of models that collectively perform the functionality of the model that was split.

The IT platform 200 may include a product inventory store 222, a user data store 226, and a collective user reviews and data store 228. In some embodiments, the one or more of the product inventory store 222, a user data store 226, and a collective user reviews and data store 228 may correspond to the data store 130 in FIG. 1.

At an initial recommendation stage, the user profile collector 202 may collect (e.g., gather, retrieve, receive) a series of inputs (sometimes referred to as, “user input data” or “user profile data”) from the user responsive to the user creating their account. In some embodiments, the initial recommendation stage takes place prior to a user implementing the recommendation and/or treatment plan generated by the IT platform 200. In some embodiments, the user profile collector 202 may collect the series of inputs from the user by sending data (e.g., instructions, code, data, questions, etc.) to the telehealth application 160 of the client device 150 to cause the telehealth application 160 to present a series of questions to the user on a display and send the user's responses to each of the questions to the user profile collector 202. In some embodiments, the data that the telehealth application 160 sends to the telehealth application 160 may include (e.g., locally stored on client device 150) the series of questions. In some embodiments, the telehealth application 160 may already have a local copy of the series of questions, such that the data sent from the telehealth application 160 is only used to instruct the telehealth application 160 to begin presenting the series of questions.

The user may provide (e.g., transmit, send) the inputs to the user profile collector 202 via the telehealth application 160 executing on the client device 150. In some embodiments, inputs may include information such as prior cannabis use, preferred products to include in the care plan (sometimes referred to as, “treatment plan” or “care program”), use preferences by day parts, diagnosed medical conditions, current medications, personality inputs, reasons for cannabis use, game results (e.g., to measure intoxication), user interaction data (e.g., images and video recordings of the user, how hard a user presses on a screen of the client device 150, eye position and movement indicative of a user focusing on one or more screens of the telehealth application 160), data from third party applications indicative of user state, such as biometric devices, health applications, mood trackers, music playlists, and other sources, and/or what the user does to relax. As discussed below, in some embodiments, the inputs to the user profile collector 202 may also be sent to the feedback processing model 220.

In some embodiments, the inputs are collected via the telehealth application 160 using multiple choice, sliders, text entry, and via open voice to text. The user profile collector 202 may store the inputs in the user's profile in the user data store 226. The user profile collector 202 may send the inputs (e.g., open voice to text and text entry inputs) to the NLP model 204 to be processed.

In some embodiments, if the user skips answering any of the questions, or does not know the answer to some questions, then the IT platform 200 may still execute (e.g., process) to produce a recommendation by using prior probabilities for those nodes and/or components.

TABLE 1 shows non-limiting examples of questions that the user profile collector 202 (and/or the telehealth application 160) may generate and present to the user of client device 150 in order to collect user input data (e.g., user profile data) at the initial recommendation stage. Each question is grouped according to a question type (e.g., Basic User Information, About User's Pain/Sleep/Anxiety (Group 1), About User's Pain/Sleep/Anxiety (Group 2), About User's Pain/Sleep/Anxiety (Group 3), etc.) and associated with a presentation method (e.g., multiple choice, location selector, NLP, etc.). In some embodiments, the user collector 202 may be a Bayesian model that is configured to generate the questions in TABLE 1. The user profile collector 202 may generate and present one or more of the examples questions to the user in any order and using any presentation method.

TABLE 1
EXAMPLE QUESTIONS FOR COLLECTING USER INPUT DATA AT
AN INITIAL RECOMMENDATION STAGE (QUESTION TYPE)

| | |
|---|---|
| Basic User Information | Gender selection (multiple choice) |
| | Age (entered text) |
| About User's | Where does it hurt? (location selector) |
| Pain/Sleep/Anxiety (Group 1) | How much does it hurt? (Stanford pain scale) |
| | Describe your pain. Is it sharp or dull? Is it constant or does it come |
| | and go? How often is the pain? (NLP) |
| | Does your pain keep you from falling asleep or wake you up at night? |
| | (NLP) |
| | How is your life being impacted? Are your activities limited? Your |
| | mood changed? (NLP) |
| | NLP output for data model: model wants to gauge how |
| | severe/urgent this issue is - also whether a mood lift may be |
| | needed in addition to addressing the primary issue |
| | Thank you. If there's anything else you'd like to share, please feel |
| | free. (NLP) |
| | Cause/diagnosis (multiple choice options related to location on body) |
| | POEM sliders relevant to pain location and diagnosis |
| | Pain meds to be replaced (check boxes with pulldowns for frequency |
| | and dose) |
| | How did (medicine previously used) work for you? Why are you |
| | looking to move on from (medicine used)? (NLP) |
| | Pain meds to be used concurrently with cannabis (check boxes with |
| | pulldowns for frequency and dose) |
| | For how long have you been feeling the pain? |
| About User's | How would you rate the quality of your sleep over the last week? |
| Pain/Sleep/Anxiety (Group 2) | (Single item Sleep Quality 1-10 Scale (SQS) slider) |
| | How often do you have trouble getting a good night of sleep? (NLP) |
| | Do you have trouble getting to sleep or staying asleep? Is sleep |
| | interrupted by outside forces? (NLP) |
| | How is your life being impacted? Are your activities limited? Your |
| | mood changed? (NLP) |
| | NLP output for data model: model wants to gauge how |
| | severe/urgent this issue is - also whether a mood lift may be |
| | needed in addition to addressing the primary issue |
| | Can you talk about your bedroom setting? Do you have a TV? Is it |
| | very dark? What about electronic usage in bed? Unwinding prior to |
| | going to sleep? (NLP) |
| | Thank you. If there's anything else you'd like to share, please feel |
| | free. (NLP) |
| | When you are in bed not sleeping, is it because you are thinking about |
| | daytime concerns? (multiple choice) |
| | Cause/diagnosis check boxes (options that could affect sleep) |
| | Depending on medical condition, does it keep you awake? |
| | Sleep meds to be replaced (check boxes with pulldowns for frequency |
| | and dose) |
| | How did (sleep medicine previously used) work for you? Why are |
| | you looking to move on from (medicine used)? |
| | Sleep meds to be used concurrently (check boxes with pulldowns for |
| | frequency and dose) |
| | Is sleep interrupted by body-related sensations (urination, pain, RLS, |
| | spasms)? (NLP) |
| About User's | How would you rate the quality of your sleep over the last week? |
| Pain/Sleep/Anxiety (Group 3) | (Slider or emoji 0-10 scale) |
| | How often do you feel anxious or stressed? (NLP) |
| | NLP output for data model: keywords showing stress |
| | frequency (all the time, only at night, once a week, etc.) |
| | Is there a time of day when you tend to feel more stressed/anxious? |
| | (multiple choice) |
| | Are you up at night thinking about the day's stressors? (multiple |
| | choice) |
| | When do you generally prefer to unwind? (multiple choice) |
| | How is your life being impacted? Are your activities limited? Your |
| | mood changed? (NLP) |
| | NLP output for data model: model wants to gauge how |
| | severe/urgent this issue is - also whether a mood lift may be |
| | needed in addition to addressing the primary issue |
| | When you feel stressed or anxious, how intense are your feelings? |
| | (scale) |
| | What do you think causes the bulk of your stress? (NLP) |
| | How do you cope with stress? (NLP) |
| | Thank you. If there's anything else you'd like to share, please feel |
| | free. (NLP) |
| | Cause/diagnosis check boxes (options related to stress/anxiety) |
| | Anxiety meds to be replaced (check boxes with pulldowns for |
| | frequency and dose) |
| | How did (anxiety medicine previously used) work for you? Why are |
| | you looking to move on from (medicine used)? |
| | Anxiety meds to be used concurrently (check boxes with pulldowns |
| | for frequency and dose) |
| About the User & Cannabis | Have you used cannabis regularly in the last 60 days? (multiple |
| | choice) |
| | How often? (multiple choice) |
| | Are you looking to replace a current cannabis regimen or add |
| | to it? (multiple choice) |
| | Why are you looking to replace a current cannabis regimen |
| | or add to it? (NLP) |
| | NLP output. Need to discern what about their |
| | current regimen they don't like/what is missing, |
| | search for key words like not strong enough, still |
| | suffering from X, drowsy, anxious, etc. |
| | What products and doses have you used most successfully in |
| | the past? (up to 3 open text field pairings) |
| | What products and doses have proved least successful in the |
| | past? (up to 3 open text field pairings) |
| | Which dispensary do you prefer to shop? |
| | Are you open to using products that cause you to feel intoxicated? |
| | (multiple choice) |
| | At which times of the day would it be unwise for you to feel |
| | intoxicated? (time sliders giving the option to indicate preference |
| | variation for intoxication at different times of day) |
| | Forms to avoid (multiple choice) |
| | How much are you willing to spend on cannabis per month? (cost |
| | slider of a range) |
| About the User | Which body type is closest to your own? (pick illustration) |
| | Do you have any of the following chronic conditions? (check boxes |
| | and open text option) |
| | Do you have any of the following allergies? (check boxes and open |
| | text option) |
| | Are you taking any of the following medications? (multiple choice) |
| | At about what time do you typically go to bed? (drop down menu) |
| | What should you know about you that will make our collaboration |
| | more successful - and feel most right? (personality and temperament |
| | check boxes) |
| | What do you most often do when you want to lift your mood? |
| | (activity check boxes) |
| | Anything else we should know that will be helpful to our |
| | collaboration? (NLP) |

The NLP model 204 receives the user input data (e.g., open voice to text and text entry inputs) from the user profile collector 202. The NLP model 204 may process the user input data into probabilistic multiple choice outputs that can be used by other models in the IT platform 200, such as the diagnosis model 206, the regimen recommendation models 208, and the feedback processing model 220. An example of an open voice input may include a question that asks the user to talk about their pain diagnosis.

The NLP model 204 may classify (e.g., group, organize) the text into predefined diagnosis categories to match the response to a specific diagnosis, and generate probabilistic values based on the classification. For example, the NLP model 204 may be X % certain that the user has arthritis, and Y % certain that the user has another condition. The NLP model 204 may then send this form of input into the diagnosis model 206 using the probabilistic values for each diagnosis category. The NLP model 204 may analyze (e.g., process) voice notes for tone, mood, stress level, etc. In some embodiments, the NLP model 204 may send the user input data (e.g., user profile data) to the diagnosis model 206.

The diagnosis model 206 may be configured to determine and select which regimen recommendation model 208 (sometimes referred to as, “malady model) would be the best fit for the user and launch (e.g., run) the selected regimen recommendation model 208. In some embodiments, the diagnosis model 205 may be a Bayesian model, which is a statistical model that uses probability to represent all uncertainty within the Bayesian model, and where the uncertainly may include the uncertainty regarding the output and the uncertainty regarding the input (e.g., parameters) to the Bayesian model. A malady model may include models that provide specific regimen recommendations for pain, sleep, anxiety, and/or other medical or wellness conditions like athletic recovery, stress relief, social enjoyment, personal fulfillment, depression, etc. In some embodiments, the diagnosis model 206 may determine certain user buckets, such as personality type, and user type based on cannabis experience. In some embodiments, the diagnosis model 206 may be included in one or more of the regimen recommendation models 208.

The diagnosis model 206 may be configured to receive the user input data (e.g., as collected by the user profile collector 202) and the probabilistic values from the NLP model 204.

The diagnosis model 206 may be configured to generate a diagnosis model output indicative of the ideal regimen recommendation model for that user, as well as the probability of varying person types, such as the probability of being “happy go lucky”, or how much of an experienced user someone is. The diagnosis model 206 may store the diagnosis model output (e.g., ideal regimen recommendation model for that user and the probability of varying person types) in the user's profile in the user data store 226. The regimen recommendation model 108 may retrieve the diagnosis model output from the user data store 126 and use as probabilistic inputs.

The IT platform 200 may train (e.g., manually or automatically) the diagnosis model 206 using research data associated with cannabis, clinical trials associated with cannabis, and/or data indicative of physician expertise and experience associated with cannabis. In some embodiments, the IT platform 200 may be configured to further train (e.g., tune) the diagnosis model 206 by matching physician recommendations to the model output. The IT platform 200 may continually re-train (e.g., update) the diagnosis model 206 using the dataset generated from one or more of the users (e.g., patients) of the intelligent telehealth platform 200, their user profile data, all their feedback inputs (sometimes referred to as, “feedback data”), the careplan attributes for recommended products by phase, and/or their progress in well-being and activity improvement metrics. In some embodiments, the IT platform 200 may estimate the one or more probabilities in each node of the diagnosis model 206 based on trial results and physician knowledge and experiences to represent the uncertainty throughout the model.

The diagnosis model 206 may include (or be associated with) a patient success node that is indicated as the target node that may be optimized. The patient success node may be based on how well that type of user does with the output from the model recommended. The outputs from the feedback processing model 220 for patient satisfaction may be used as inputs for this model to provide evidence for the patient success node. For example, a user may have both anxiety and pain, and which regimen recommendation model 208 to run may be less certain between those two. When data is used from similar users, the IT platform 200 may optimize for the patient success node and be able to determine which model works more frequently for these types of users. The IT platform 200 may find that the pain model may be better suited for older users who suffer from both pain and anxiety, while younger users may benefit more from addressing the anxiety first.

The IT platform 200 may use parameter estimation and/or parameter updating to update the model. Parameter estimation may be used to update the probability weightings within the nodes to optimize for patient satisfaction. Parameter updating may be used to update the connections between the nodes to also optimize for patient satisfaction. In some embodiments, certain nodes within the model can be replaced by machine learning (ML). For example, whether or not a user is “happy go lucky” can be determined outside of the Bayesian model using ML techniques, and the output of that can populate the “happy go lucky” diagnosis directly. ML techniques could include regression, classification, clustering, dimensionality reduction, neural nets and deep learning, reinforcement learning, natural language processing, association models, causation models, reinforced learning, etc. These models may replace the Bayesian models, or they may run outside of the Bayesian models and generate outputs that can be used as inputs to the Bayesian.

The regimen recommendation model 209 may be configured as a Bayesian model that generates (outputs) all of the care plan attributes. For each day, sessions are recommended to fill the day parts. Each session may have a start time, recommended product types (e.g., form and cannabidiol:tetrahydrocannabinol (CBC:THC) ratio), recommended doses, and/or recommended use type (e.g., directed versus optional). The product form is output in probabilities, so that the highest recommended form has the highest probability. Any forms the model recommends completely avoiding are given a probability of 0.

As discussed below, the regimen recommendation model 209 updates its recommendation when new feedback inputs are provided. The inputs for the feedback are processed in the feedback processing model 220. The new recommendation is used to generate an updated careplan draft for the next phase of the process. In some embodiments, the feedback processing model 220 may also ingest one or more of the inputs (as discussed herein) that are ingested by the user profile collector 202.

The IT platform 200 may train the regimen recommendation model 209 using research data associated with cannabis, clinical trials associated with cannabis, and/or data indicative of physician expertise and experience associated with cannabis. In some embodiments, the IT platform 200 may be configured to further train (e.g., tune) the regiment recommendation model 208 by matching physician recommendations to the model output. The IT platform 200 may continually re-train (e.g., update) the recommendation model 209 using the dataset generated from one or more of the users of the intelligent telehealth platform 200, their user profile data, all their feedback inputs, the careplan attributes for recommended products by phase, and/or their progress in well-being and activity improvement metrics. In some embodiments, the IT platform 200 may estimate the one or more probabilities in each node of the recommendation model 209 based on trial results and physician knowledge and experiences to represent the uncertainty throughout the model.

The recommendation model 209 may include (or be associated with) a patient success node that is indicated as the target node that may be optimized. The patient success node may be based on patient well-being metrics, progress on their activities of daily living (ADLs) (e.g., which is output by the feedback processing model 220), specifics of each phase of their careplan, how each phase was modified, and/or how the update worked for the user.

The IT platform 200 may use parameter estimation and/or parameter updating to update the model. Parameter estimation may be used to update the probability weightings within the nodes to optimize for patient satisfaction. Parameter updating may be used to update the connections between the nodes to also optimize for patient satisfaction. In some embodiments, certain nodes within the model can be replaced by machine learning (ML). For example, whether or not a user is “happy go lucky” can be determined outside of the Bayesian model using ML techniques, and the output of that can populate the “happy go lucky” diagnosis directly.

The inventory model 210 may include all the products in the product pool (e.g., in the product inventory storage 222). The inventory model 210 may tag each product for attributes related to that product. In some embodiments, a tag may include CBD:THC type, other dominant cannabinoids, dominant terpenes, dispensary availability, product descriptions, whether or not the product is full spectrum or not, allergens, form, etc.

The inventory model 210 may automatically tag (e.g., assign) the inventory for dose conversions, so that each product is able to be presented in an easy to follow dose (e.g., drops, gummies, squares of chocolate, etc.) instead of a milligram (mg) amount.

The inventory model 210 may tag the individual products in the inventory with collective user scores. Each product that has been recommended and used is scored by the inventory model 210 as collective user feedback data comes in for effectiveness for varying maladies and for typical feeling states induced. In some embodiments, these scores include pain_relief_score, sleep_score, etc. They are also scored based on how well tolerated they are at different times of the day.

The inventory model 210 may be a machine learning model, a Bayesian model, or a rules-based model. The inventory model 210 may consist of ranking and filtering through the inventory to find a product that matches the attributes that were output from the regimen recommendation model and scores the highest based on the review score tags. The inventory model 210 was developed using physician guidance for determining the ranking and filtering logic.

The inventory model 210 uses product scores and other inputs to select products for the user. The inventory model 210 selects the closest dispensary to the user that matches the medical card status. The inventory model 210 then ranks and filters (e.g., based on user and care plan attribute information from the regimen recommendation model 208) the products from the selected dispensary. The inventory model 210 filters out products based on allergens, THC type, and/or forms that have a probability of 0.

The inventory model 210 then ranks (e.g., prioritizes, orders, sorts) the remaining products based on their scores for malady (e.g., disease, pain, sleep, anxiety), day part score, clarity, and/or energy, etc. The inventory model 210 selects the highest scoring product for the user for each product role.

The instructions model 212 may be a machine learning model, a Bayesian model, or a rules-based model. The inputs of the instructions model 212 may include the specific product information included in the recommendation, the dosages recommended, as well as user profiling inputs like medical conditions, a wellness condition, use preferences, user experience, history with certain cannabis forms, past side effects, set and setting history and feedback, other medication usage, and/or other feedback or profiling inputs provided. The output of the instructions model 212 is user-specific product use instructions, expected effect onset and durations for each session, set and setting recommendations, general guidance, as well as specific use instructions given a user's incoming physical and mental state or situation, and any relevant session warnings based on a user's specific profile (e.g., user input profile).

The instructions model 212 runs ahead of the careplan approval, and provides instructions for use given the status of the user. Additionally, instructions for use are also provided assuming different potential states of the user. This could include things like higher pain than normal, an extra stressful situation, etc. Modified use instructions are then provided and approved based on these situations, so that should they arise, the instructions model 112 can update the instructions in real-time. For example, if the user is in more pain than usual, a higher dose of a product may be recommended. This model was developed using a combination of available research data and medical expertise from cannabis physicians.

The why recommended model 224 may be a machine learning model, a Bayesian model, or a rules-based model. The why recommended model 224 is configured to generate a model output describing reasons for the product selections and use times that were made. The inputs to the why recommended model 224 may include one or more of the following: user profile data (e.g., collected from both profiling and feedback); information about the user's preferences regarding form, mental clarity, desired energy, and relaxation by day; information about the user's experience with cannabis; information about different medical conditions a user (which may also drive the recommendation for certain product types); the products selected for the user; and/or the outputs from the diagnosis model 206 regarding person type.

The outputs of the why recommended model 224 are divided into categories that include dosing, product mix, bedtime, accessories, and/or cost. Each output is a string of copy that populates the “Why Recommended” section of the telehealth application 160. The specific copy that is output is determined by the recommended model 224.

The IT platform 200 may train (e.g., manually or automatically) the why recommended model 224 using research data associated with cannabis, clinical trials associated with cannabis, and/or data indicative of physician expertise and experience associated with cannabis. In some embodiments, the IT platform 200 may be configured to further train (e.g., tune) the why recommended model 224 by matching physician recommendations to the model output. The IT platform 200 may continually re-train (e.g., update) the why recommended model 224 using the dataset generated from one or more of the users of the intelligent telehealth platform 200, their user profile data, all their feedback inputs, the careplan attributes for recommended products by phase, and/or their progress in well-being and activity improvement metrics. In some embodiments, the IT platform 200 may estimate the one or more probabilities in each node of the why recommended model 224 based on trial results and physician knowledge and experiences to represent the uncertainty throughout the model.

As the user uses the products (e.g., during treatment) and follows the careplan that was produced by the IT platform 200, the feedback collector 216 may collect a series of inputs (sometimes referred to as, “feedback inputs” or “feedback data”) from the user. In some embodiments, the feedback collector 216 may collect the series of feedback inputs from the user by sending data (e.g., instructions, code, data, questions, etc.) to the telehealth application 160 of the client device 150 to cause the telehealth application 160 to present a series of questions to the user on a display and send the user's responses to each of the questions to the feedback collector 216. In some embodiments, the data that the telehealth application 160 sends to the telehealth application 160 may include (e.g., locally stored on client device 150) the series of questions. In some embodiments, the telehealth application 160 may already have a local copy of the series of questions, such that the data sent from the telehealth application 160 is only used to instruct the telehealth application 160 to begin presenting the series of questions.

The user may provide (e.g., transmit, send) the feedback inputs to the feedback collector 216 via the telehealth application 160 executing on the client device 150. In some embodiments, feedback inputs may include information related to pain level before and during use, mood, ADL performance, well-being, and/or sleep quality, etc. In some embodiments, the feedback inputs are collected via the telehealth application 160 using multiple choice, sliders, text entry, and via open voice to text. The feedback collector 216 may store the feedback inputs in the user's profile in the user data store 226.

TABLE 2 shows non-limiting examples of questions that the feedback collector 216 (and/or the telehealth application 160) may generate and present to the user of client device 150 in order to collect feedback inputs about the user's experience using the product and following the careplan. Each question is grouped according to a time period in which the questions are asked and associated with a presentation method (e.g., multiple choice, location selector, NLP, etc.). In some embodiments, the feedback collector 216 may be a Bayesian model that is configured to generate the questions in TABLE 2. The feedback collector 216 may present one or more of the examples questions to the user in any order and using any presentation method.

TABLE 2
EXAMPLE QUESTIONS FOR COLLECTING FEEDBACK INPUTS
DURING AND/OR AFTER TREATMENT (QUESTION TYPE)

| | |
|---|---|
| Checking-In Feedback | It's been about [time period] since you used your drops. How's your |
| (i.e., at some point during the | pain level now? (Stanford Pain Scale Slider) |
| course of treatment) | Describe any side effects or notable pleasant/unpleasant sensations? |
| | (NLP) |
| | NLP output for data model: search for side effect keywords |
| | (dry eyes, paranoia, too intense, anxiety, sleepy, etc.) as well |
| | as pleasant sensation keywords (elation, euphoria, relaxed, |
| | giggly, etc.) |
| End of Session Feedback | It's been about [time period we expect to conclude the session] since |
| | you used your drops. How's your pain level now? (Stanford Pain |
| | Scale Slider) |
| | Rate your session (1-5 stars) |
| | Provide a short review of your session. (NLP) |
| | NLP output for data model: if session had low stars, search |
| | for keywords like felt nothing, didn't work, etc. Model will |
| | also track the output of the stress level in the user's voice |
| | compared to their stress level prior to starting to be used in |
| | combination with the star rating. |
| End of Day Feedback | Reflect back. How satisfied were you with your experience of |
| | cannabis today? Consider effectiveness in managing your pain and |
| | side effects, in particular. (slider) |
| | Throughout the day, about how often did you feel: (WHO Well-Being |
| | Scale sliders for each) |
| | Cheerful & in good spirits? |
| | Calm & relaxed? |
| | Attentive & engaged? |
| | Energetic & vigorous? |
| | Anything else you'd like to share about today? Any unusual feeling, |
| | highs, or lows? (NLP) |
| | NLP output for data model: model will track keywords for |
| | highs and lows and trend over time. Model will also use the |
| | output of stress level from user's voice and track this trend |
| | over time. |

When a user completes a telemedicine session, the telemed session data collector 218 collects the telemedicine session data and automatically enters the telemedicine session data into an administrator (“admin”) portal of the IT platform 200. In some embodiments, the telemed session data collector 218 may present the telemedicine session data on a display, at which point, a physician may manually enter the telemedicine session data directly into the admin portal in the form of multiple choice inputs. For example, the patient may note that one of their products make them too tired, and the physician can then enter that side effect for that product into the admin portal. These inputs are then able to be used by the feedback processing model 220 to provide more evidence for an updated careplan.

The feedback processing model 220 is a Bayesian model that is configured to process all the feedback data collected from the user. This includes the in-session feeling indications, as well as weekly survey results where they indicate how each product is working and how the plan is working for them in general. Additional feedback from the telemed session data collector 218 may also be included if available. Even if the IT platform 200 determines that there is missing (or no) data, then the model may still decide to run without it. The model will incorporate as much or as little feedback as is available, using prior probabilities (e.g., associated with a prior run of the model) when feedback data is missing. The model is designed with specific nodes and probabilities in order to weigh all the available evidence and determine the update to the plan that is needed, with a degree of certainty. Outputs include nodes such as THC adjustment (whether or not to increase or decrease the THC for a given feeling state), dose adjustment (whether or not the recommended dose for a given feeling state should increase or decrease), and/or target strain. The probabilistic outputs of this model are then used as additional inputs into the regimen recommendation model 208 to provide an updated recommendation. The feedback processing model 220 computes overall progress and satisfaction, as well as the most likely adjustments needed to the careplan. Although FIG. 2 shows the feedback processing model 220 as a single model, in some embodiments, the feedback processing model 220 may include a plurality of feedback processing models 220 that are interconnected to process all the feedback data collected from the user.

The IT platform 200 may train the feedback processing model 220 using research data associated with cannabis, clinical trials associated with cannabis, and/or data indicative of physician expertise and experience associated with cannabis. In some embodiments, the IT platform 200 may be configured to further train (e.g., tune) the feedback processing model 220 by matching physician recommendations to the model output. The IT platform 200 may continually re-train (e.g., update) the feedback processing model 220 using the dataset generated from one or more of the users of the intelligent telehealth platform 200, their user profile data, all their feedback inputs, the careplan attributes for recommended products by phase, and/or their progress in well-being and activity improvement metrics. In some embodiments, the IT platform 200 may estimate the one or more probabilities in each node of the feedback processing model 220 based on trial results and physician knowledge and experiences to represent the uncertainty throughout the model.

The feedback processing model 220 may include (or be associated with) a patient success node that is indicated as the target node that may be optimized. The patient success node may be based on patient well-being metrics, progress on their activities of daily living (ADLs) (e.g., which is output by the feedback processing model 220), specifics of each phase of their careplan, how each phase was modified, and/or how the update worked for the user.

The IT platform 200 may use parameter estimation and/or parameter updating to update the model. Parameter estimation may be used to update the probability weightings within the nodes to optimize for patient satisfaction. Parameter updating may be used to update the connections between the nodes to also optimize for patient satisfaction. In some embodiments, certain nodes within the model can be replaced by machine learning (ML). For example, whether or not a user is “happy go lucky” can be determined outside of the Bayesian model using ML techniques, and the output of that can populate the “happy go lucky” diagnosis directly.

The IT platform 200 tags the inventory in the product inventory store 222 by time of day use scores, feeling state use scores (e.g., energy, mental clarity, relaxation, etc.), malady score (e.g., disease, pain, anxiety, sleep, etc.), CBD:THC type, form, allergens, cannabinol (CBN), cannabigerol (CBG), cannabichromene (CBC), cannabidiol (CBD), etc.), terpenes, dose to product conversions, etc.

The IT platform 200 may use the collective user reviews and data (e.g., from the collective user reviews and data store 228) to tag the inventory in the product inventory store 222. The IT platform 200 may use the scores (e.g., time of day use score, feeling state use score, malady score) to generate a recommendation.

The final careplan generator 224 is configured to send the final careplan to the telehealth application 160 of the client device 150 to cause the telehealth application 160 to present the final careplan to the user on a display. The final careplan it presented to the user after both profiling and after each phase, and includes the recommended products, dosages, times of day for use, instructions, and copy explaining why the IT platform 200 chose the products in the final careplan. In addition, the user is able to view their progress at any time through their patient progress portal.

The user data store 226 is configured to store the user profile data for all users of the IT platform 200. Each of the models of the IT platform 200 are able to store their respective data in the user data store 226. User profile data may include every piece of data the IT platform 200 gathers about a user, such as all of the data the user provides during the profile phase, all of the feedback data the user provides during and after treatment, the user's progress toward well-being, all of the products they've used, doses, use times, etc. and how effective the products have been at making the user feel how they want to feel. In some embodiments, the IT platform 200 may organize the data in the user data store 226 into an ontology to improve the efficiency in which the IT platform 200 can retrieve the data from the user data store 226.

The collective user reviews and data store 228 is configured to store all of the user feedback and reviews into a dataset that can be used to tag the inventory. Each time a user leaves feedback on a certain product, that feedback is processed by the feedback processing model 220 to output a score for that product based on attributes like pain, energy, time of day use, etc. The resulting score from that user is added to the dataset of existing scores for that product, and the average/median score is used to tag the inventory. The data is also organized by person type and other diagnoses, so each person type may have a different score for relaxation, for example.

Prior to the final careplan being sent to the user, the instructions model 212 may send the careplan to a physician review 230 (e.g., physicians, medical group) to review and approve all aspects of the careplan (e.g., regimen, products, doses, why recommended copy, instructions, use times, patient feedback, etc.) and modify the careplan, if needed. The physician review 230 may then send the approved careplan to the final careplan generator 224.

As discussed herein, a Bayesian model may be used in place of a rules-based model to provide several benefits. A first benefit is that a Bayesian model allows the IT platform 200 to run the Bayesian model with an incomplete dataset. For example, the IT platform 200 may be missing one or more datapoints from a user (e.g., a user may miss one or more sessions, fail to provide feedback, or not know responses to some of the questions asked during the profiling phase), but the Bayesian model can use assume priors to put a reasonable guess into what that datapoint would be. While, the overall uncertainty may increase, the Bayesian model would still be able to produce a result. Additionally, as data is collected, these priors become better informed, and can actually decrease the amount of upfront information the IT platform 200 would need to collect from a user. This is especially important given the nature of the NLP inputs that the IT platform 200 collects, as there could be a higher likelihood of not receiving complete answers.

A second benefit is the scaling ability when using a Bayesian model. Instead of hard cut-offs like in rules, a Bayesian approach puts a value of certainty to each input. The certainties for all the inputs are then combined to produce a decision. Each decision is weighted, so that the best and second-best decisions can be seen clearly. This creates a much more flexible platform, and allows for better-informed adjustments when the IT platform 200 receives user feedback.

A third benefit is the easy addition or deletion of certain variables. Since utility values are all combined using separate nodes, variables can be added or removed relatively easily, compared to a rules-based platform which would likely require rewriting many of the rules. This provides flexibility on the front end.

A fourth benefit is that IT platform 200 (or administrator) may have greater visibility into the activities of each of the models, such that if there is an unexpected output, then the IT platform 200 may quickly identify the model causing the unexpected output. This also gives the IT platform 200 the ability to generate data that is indicative of the reasons for a given recommendation.

A fifth benefit is that once data becomes available, then the same network can be updated based on the data. This allows the same network to be used instead of the need to develop a new machine learning model. It also allows the IT platform 200 (or administrator) to maintain control over the network. For example, the IT platform 200 can limit the effect of the data on the network if the IT platform 200 has a partial dataset (e.g., less than desired).

FIGS. 3A-3N depict various example graphical user interfaces of the telehealth application 160 in FIG. 1, according to some embodiments. In some embodiments, the telehealth application 160 may be configured to execute on the host system 110, where the output of the execution is presented on a screen of the host system 110. In some embodiments, the telehealth application 160 may be configured to execute on the host system 110, where the output of the execution is sent to the client device 150 via the network 105 to cause the client device 150 to display the output on a screen of the client device 150. In some embodiments, some screens of the graphical user interface (GUI) may only be viewable by an administrator of the IT platform 200, while other screens may only viewable by a user (e.g., a patient) using the IT platform 200. In some embodiments, the telehealth application 160 may be a web browser.

FIG. 3A is an example graphical user interface of the telehealth application 160 depicting a method for displaying a list of dispensaries stored in a storage of the IT platform 200, according to some embodiments. As shown in the GUI 300a, the dispensaries are either tagged as local, meaning they have a physical store, or online, meaning anyone can order the products online. An administrator of the IT platform 200 may use the GUI 300a to upload new dispensaries to the IT platform 200 via a file (e.g., a table, a mapping file, a comma separated value (.CSV) file) to cause the IT platform 200 to store the new dispensaries in a storage of the IT platform 200.

FIGS. 3B-3C are example graphical user interfaces of the telehealth application 160 depicting a method for displaying a list of products stored in a storage of the IT platform 200, as well as the details of each of the products, according to some embodiments. An administrator of the IT platform 200 may use the GUI 300b to upload new products to the IT platform 200 via a file (e.g., a table, a mapping file, a comma separated value (.csv) file) to cause the IT platform 200 to store the new products in a storage of the IT platform 200. As shown in the GUI 300b, the IT platform 200 tags products for things like category, cannabinoid type, brand, product name and image, dispensary location, and/or cost. As shown in the GUI 300c, clicking on a product reveals more product details such as allergens, description, total mg, accessories required, etc. The product file (e.g., csv file) may also contain more detail such as day part score, malady score, specific amounts of cannabinoids, full spectrum, etc.

FIGS. 3D-3E are example graphical user interfaces of the telehealth application 160 depicting a method for displaying a list of patient profiles stored in a storage of the IT platform 200, as well as the details of each of the patient profiles, according to some embodiments. An administrator of the IT platform 200 may use the GUI 300d to upload new patient profiles to the IT platform 200 via a file (e.g., a table, a mapping file, a comma separated value (.csv) file) to cause the IT platform 200 to store the new patient profiles in a storage of the IT platform 200. The patient profiles are filterable by the next action needed, as well as by the specific patient info. When the user needs to have a careplan created, a pending tag is shown. When the user needs their plan reviewed again because they are nearing the end of the phase, a review phase tag is shown. In some embodiments, a tag (e.g., a red tag) may indicate that feedback data from a user may need attention.

As shown in GUI 300e, the detail tab contains all of the profiling detail, including all the open-ended responses and medical information from the patient. This is also edit-able, and some of the open-ended responses are translated into multiple choice inputs for the model. Once the profile is ready, a user (or administrator) of the IT platform 200 may create a careplan by clicking the Create Careplan button, which causes one or more components (e.g., models) of the IT platform to ingest the user profile data (and other sets of data discussed herein) to create a draft careplan.

FIG. 3F is an example graphical user interfaces of the telehealth application 160 depicting a method for displaying the model outputs from each of the models of the IT platform 200, according to some embodiments. As shown, the model outputs are organized into an ontology for more organized tracking of the dataset created.

FIGS. 3G-3H are example graphical user interfaces of the telehealth application 160 depicting a method for displaying a draft careplan created by the models of the IT platform 200, according to some embodiments. Specifically, GUI 300g shows an example phase 1 of a draft careplan and GUI 300h shows an example phase 2 of the draft careplan. The IT platform 200 model outputs a draft careplan, filling in the day parts with the recommended product types and doses. Each dose is also converted automatically to a dose the user can understand (e.g., drops, squares of chocolate, gummies, etc.). The IT platform 200 may filter out the products of the careplan based a matching product type. The IT platform 200 may filter out the products from the careplan that are associated with allergies that match the user's allergies (as learned during the patient profiling phase). The IT platform 200 sorts the products by probability of the best matching product, and the It platform 200 automatically selects the top (e.g., best matching that of the user's profile data, etc.) product from the list.

FIG. 3I is an example graphical user interface of the telehealth application 160 depicting a method for displaying product specific instructions, according to some embodiments. For example, GUI 300I may display duration, onset, and general use instructions, as well as product-specific use instructions for each product.

FIG. 3J is an example graphical user interface of the telehealth application 160 depicting a method for displaying the model output of the why recommendation model 114 in FIG. 1, according to some embodiments. The IT platform 200 may transmit the draft careplan to a physician to allow the physician to edit and/or approve the draft careplan.

FIG. 3K is an example graphical user interface of the telehealth application 160 depicting a method for displaying a user's shopping list, according to some embodiments. The user's shopping list can be viewed and edited, as well as a preview of their entire care plan by day part.

FIG. 3L is an example graphical user interface of the telehealth application 160 depicting a method for displaying a draft careplan created by the models of the IT platform 200, according to some embodiments. That is, once the plan is ready to go, IT platform 200 (or administrator) may send the draft careplan to a physician (physician review 230 in FIG. 2) for the physician to review the draft careplan, and make edits as needed. When the physician approves the draft careplan, then the user is notified (e.g., via email, phone, text, etc.) that their plan has been finalized/approved, and that their shopping list is ready.

FIG. 3M is an example graphical user interface of the telehealth application 160 depicting a method for displaying a user's progress in completing the careplan, according to some embodiments. The GUI 300g shows the portions of the careplan that the use has completed, and the portion of the careplan that are pending.

FIG. 3N is an example graphical user interface of the telehealth application 160 depicting a method for displaying a notes session to track any communication with the patient, survey data, or physician telemed notes, according to some embodiments. The data in the notes session may be fed back and added to the feedback data that is gathered by the feedback collector 216. In some embodiments, the telemed notes may be entered automatically via multiple choice selection by the physician while on the call with the patient.

FIG. 4 is a flow diagram depicting a method for using the intelligent telehealth platform 200 to generate a recommended treatment plan to treat a malady of a user, according to some embodiments. Method 400 may be performed by processing logic that may comprise hardware (e.g., circuitry, dedicated logic, programmable logic, a processor, a processing device, a central processing unit (CPU), a system-on-chip (SoC), etc.), software (e.g., instructions and/or an application that is running/executing on a processing device), firmware (e.g., microcode), or a combination thereof. In some embodiments, method 400 may be performed by a host system, such as host system 110 in FIG. 1. In some embodiments, method 400 may be performed by an IT platform, such as IT platform 200 in FIG. 2. In some embodiments, method 400 may be performed by a client device, such as client device 150 in FIG. 1.

With reference to FIG. 4, method 400 illustrates example functions used by various embodiments. Although specific function blocks (“blocks”) are disclosed in method 400, such blocks are examples. That is, embodiments are well suited to performing various other blocks or variations of the blocks recited in method 400. It is appreciated that the blocks in method 400 may be performed in an order different than presented, and that not all of the blocks in method 400 may be performed.

As shown in FIG. 4, the method 400 includes the block 402 of obtaining a user profile dataset associated with a user. The method 400 includes the block 404 of selecting, based on the user profile dataset, a first machine learning model from a set of machine learning models respectively trained to predict cannabis treatment responses. The method 400 includes the block 406 of generating, by a processing device, a recommended treatment plan for using one or more cannabis products to treat a malady of the user based on the user profile dataset and the first machine learning model. The method 400 includes the block 408 of transmitting the recommended treatment plan to a client device to cause the client device to display the recommended treatment plan.

FIG. 5 is a block diagram of an example computing device 500 that may perform one or more of the operations described herein, in accordance with some embodiments. Computing device 500 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 500 may include a processing device (e.g., a general purpose processor, a PLD, etc.) 502, a main memory 504 (e.g., synchronous dynamic random access memory (DRAM), read-only memory (ROM)), a static memory 506 (e.g., flash memory and a data storage device 518), which may communicate with each other via a bus 530.

Processing device 502 may be provided by one or more general-purpose processing devices such as a microprocessor, central processing unit, or the like. In an illustrative example, processing device 502 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 502 may also comprise one or more special-purpose processing devices such as an application specific integrated circuit (ASIC), a field programmable gate array (FPGA), a digital signal processor (DSP), network processor, or the like. The processing device 502 may be configured to execute the operations described herein, in accordance with one or more aspects of the present disclosure, for performing the operations and steps discussed herein.

Computing device 500 may further include a network interface device 508 which may communicate with a communication network 520. The computing device 500 also may include a video display unit 510 (e.g., a liquid crystal display (LCD) or a cathode ray tube (CRT)), an alphanumeric input device 512 (e.g., a keyboard), a cursor control device 514 (e.g., a mouse) and an acoustic signal generation device 516 (e.g., a speaker). In one embodiment, video display unit 510, alphanumeric input device 512, and cursor control device 514 may be combined into a single component or device (e.g., an LCD touch screen).

Data storage device 518 may include a computer-readable storage medium 528 on which may be stored one or more sets of instructions 525 that may include instructions for one or more components and/or applications 542 (e.g., IT component 125 in FIG. 1) for carrying out the operations described herein, in accordance with one or more aspects of the present disclosure. Instructions 525 may also reside, completely or at least partially, within main memory 504 and/or within processing device 502 during execution thereof by computing device 500, main memory 504 and processing device 502 also constituting computer-readable media. The instructions 525 may further be transmitted or received over a communication network 520 via network interface device 508.

While computer-readable storage medium 528 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

Unless specifically stated otherwise, terms such as “obtaining,” “selecting,” “generating,” “transmitting,” or the like, refer to actions and processes performed or implemented by computing devices that manipulates and transforms data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein are meant as labels to distinguish among different elements and may not necessarily have an ordinal meaning according to their numerical designation.

Examples described herein also relate to an apparatus for performing the operations described herein. This apparatus may be specially constructed for the required purposes, or it may comprise a general purpose computing device selectively programmed by a computer program stored in the computing device. Such a computer program may be stored in a computer-readable non-transitory storage medium.

The methods and illustrative examples described herein are not inherently related to any particular computer or other apparatus. Various general purpose systems may be used in accordance with the teachings described herein, or it may prove convenient to construct more specialized apparatus to perform the required method steps. The required structure for a variety of these systems will appear as set forth in the description above.

The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. § 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

The foregoing description, for the purpose of explanation, has been described with reference to specific embodiments. However, the illustrative discussions above are not intended to be exhaustive or to limit the present disclosure to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the present disclosure is not to be limited to the details given herein, but may be modified within the scope and equivalents of the appended claims.

What is claimed is:

1. A method comprising:
obtaining a user profile dataset associated with a user;
selecting, based on the user profile dataset, a first machine learning model from a set of machine learning models respectively trained to predict cannabis treatment responses;
generating, by a processing device, a recommended treatment plan for using one or more cannabis products to treat a malady of the user based on the user profile dataset and the first machine learning model; and
transmitting the recommended treatment plan to a client device to cause the client device to display the recommended treatment plan.

2. The method of claim 1, wherein selecting, based on the user profile dataset, the first machine learning model from the set of machine learning models comprises:
providing the user profile dataset to a second machine learning model trained to diagnose maladies associated with at least one of pain, anxiety, or sleep patterns, wherein the second machine learning model is trained with at least one of research data associated with cannabis, clinical trial data associated with cannabis, or data indicative of physician expertise and experience associated with cannabis.

3. The method of claim 2, wherein the second machine learning model is further trained to determine a personality type of the user based on the user profile dataset, wherein the user profile dataset is indicative of at least one medical condition, a wellness condition, a cannabis use preference, and a user experience with cannabis.

4. The method of claim 1, wherein the set of machine learning models each correspond to a Bayesian model.

5. The method of claim 1, further comprising:
maintaining, in a storage, a plurality of association between a plurality of cannabis product identifiers and a plurality of scores, wherein each score comprises a tolerance score, a feeling state use score, or a malady score.

6. The method of claim 1, further comprising:
providing the user profile dataset to a second machine learning model trained to generate model data for the recommended treatment plan, wherein the model data comprises at least one of an instruction for using the one or more cannabis products, data indicative of an expected effect onset and duration for the one or more cannabis products, or a warning for using the one or more cannabis products.

7. The method of claim 1, further comprising:
providing the user profile dataset to a second machine learning model trained to generate model data for the recommended treatment plan, wherein the model data comprises the one or more reasons the first machine learning model selected the one or more cannabis products to treat the malady of the user.

8. The method of claim 1, further comprising:
receiving feedback data indicative of an experience of the user when using the one or more cannabis products based on the recommended treatment plan;
determining that the feedback data is incomplete; and
generating the recommended treatment plan even though the feedback data is incomplete.

9. The method of claim 8, further comprising:
obtaining uncertainty data associated with a prior iteration of the first machine learning model, wherein the generating the recommended treatment plan is further based on the uncertainty data.

10. The method of claim 1, further comprising:
receiving physician approval of the recommended treatment plan prior to transmitting the recommended treatment plan to the client device.

11. An apparatus comprising:
a memory; and
a processing device, operatively coupled to the memory, to:
obtain a user profile dataset associated with a user;
select, based on the user profile dataset, a first machine learning model from a set of machine learning models respectively trained to predict cannabis treatment responses;
generate, by a processing device, a recommended treatment plan for using one or more cannabis products to treat a malady of the user based on the user profile dataset and the first machine learning model; and
transmit the recommended treatment plan to a client device to cause the client device to display the recommended treatment plan.

12. The apparatus of claim 11, wherein the processing device is further to:
provide the user profile dataset to a second machine learning model trained to diagnose maladies associated with at least one of pain, anxiety, or sleep patterns, wherein the second machine learning model is trained with at least one of research data associated with cannabis, clinical trial data associated with cannabis, or data indicative of physician expertise and experience associated with cannabis.

13. The apparatus of claim 12, wherein the second machine learning model is further trained to determine a personality type of the user based on the user profile dataset, wherein the user profile dataset is indicative of at least one medical condition, a wellness condition, cannabis use preference, and a user experience with cannabis.

14. The apparatus of claim 11, wherein the set of machine learning models each correspond to a Bayesian model.

15. The apparatus of claim 11, wherein the processing device is further to:
maintain, in a storage, a plurality of association between a plurality of cannabis product identifiers and a plurality of scores, wherein each score comprises a tolerance score, a feeling state use score, or a malady score.

16. The apparatus of claim 11, wherein the processing device is further to:
provide the user profile dataset to a second machine learning model trained to generate model data for the recommended treatment plan, wherein the model data comprises at least one of an instruction for using the one or more cannabis products, data indicative of an expected effect onset and duration for the one or more cannabis products, or a warning for using the one or more cannabis products.

17. The apparatus of claim 11, wherein the processing device is further to:
provide the user profile dataset to a second machine learning model trained to generate model data for the recommended treatment plan, wherein the model data comprises the one or more reasons the first machine learning model selected the one or more cannabis products to treat the malady of the user.

18. The apparatus of claim 11, wherein the processing device is further to:
receive feedback data indicative of an experience of the user when using the one or more cannabis products based on the recommended treatment plan;
determine that the feedback data is incomplete; and
generate the recommended treatment plan even though the feedback data is incomplete.

19. The apparatus of claim 8, wherein the processing device is further to at least one of:
obtain uncertainty data associated with a prior iteration of the first machine learning model, wherein the generating the recommended treatment plan is further based on the uncertainty data; or
receive physician approval of the recommended treatment plan prior to transmitting the recommended treatment plan to the client device.

20. A non-transitory computer-readable storage medium including instructions that, when executed by a processing device of a host system, cause the processing device to:
obtain a user profile dataset associated with a user;
select, based on the user profile dataset, a first machine learning model from a set of machine learning models respectively trained to predict cannabis treatment responses;
generate, by the processing device, a recommended treatment plan for using one or more cannabis products to treat a malady of the user based on the user profile dataset and the first machine learning model; and
transmit the recommended treatment plan to a client device to cause the client device to display the recommended treatment plan.
